(19) United States
(12) Patent Application Publication
(10) Pub. No.: US 2008/0075776 A1
(43) Pub. Date: Mar. 27, 2008

(54) Topical Pharmaceutical Compositions Containing An Antiacne Compound And Antibiotic Compound

(75) Inventors: Ghanshyam Narayan Chaudhari, Nashik (IN); Vasant Sitaram Khachane, Nashik (IN); Nitin Babulal Bhamre, Nashik (IN); Jagannath Macharla, Karnataka (IN)

Correspondence Address: M. CARMEN & ASSOCIATES, PLLC, 170 OLD COUNTRY ROAD, SUITE 400, MINEOLA, NY, 11501, US

(73) Assignee: GLENMARK PHARMACEUTICALS LIMITED, ANDHERI (EAST), MUMBAI - 400 099 (IN)

(21) Appl. No.: 11/667,283

(22) PCT Filed: Nov. 8, 2005
(86) PCT No.: PCT/IB05/03302

§ 371 Date: Sep. 13, 2007

Related U.S. Application Data
(60) Provisional application No. 60/625,872, filed on Nov. 8, 2004.

(51) Int. Cl. A61K 9/00; A61K 31/4025; A61P 17/10

(52) U.S. Cl. 424/484; 514/422

(57) ABSTRACT

Pharmaceutical compositions suitable for the treatment of skin disorder are provided which include at least a therapeutically effective amount of at least on antiacne agent and at least one antibiotic agent as active pharmaceutical ingredients and a hydrophilic matrix capable of providing a constant and uniform release of the active pharmaceutical ingredients.

### PRIORITY

This application claims the benefit under 35 U.S.C. §119 to Provisional Application No. 60/625,872, filed Nov. 8, 2004 and entitled “TOPICAL PHARMACEUTICAL COMPOSITIONS CONTAINING AN ANTIACNE COMPOUND AND ANTIBIOTIC COMPOUND”, the contents of which are incorporated by reference herein.

### BACKGROUND OF THE INVENTION

1. Technical Field

The present invention relates generally to pharmaceutical compositions suitable for the treatment of bacterial dermatological infections and processes for their preparation. More specifically, the present invention is directed to topical pharmaceutical combinations containing at least an antiacne agent and an antibiotic agent.

2. Description of the Related Art

Acne vulgaris is an inflammatory disease of the sebaceous glands characterized by an eruption of the skin, often pustular in nature but not suppurative. Acne is a common affliction of an adolescent and also affects a small but significant percentage of the adult population. Acne may result in unsightly lesions, particularly on the face, and in some cases may even cause severe scarring.

There are a variety of methods for treating acne vulgaris such as, for example, administering various agents either orally or topically to the skin. Nevertheless, acne vulgaris is seldom cured and only can be controlled with difficulty. In no case has a treatment designed for any of the aforementioned causes proven to be uniformly effective. One treatment that is believed to be proven effective is the oral administration of isotretinoin (Accutane®). This medication, however, has numerous side effects, one being its potential to induce severe birth defects.

In general, topical treatment for an acne related conditions includes a wide variety of topical compositions such as, for example, creams, lotions and solutions. Typically, these products have a single ingredient selected from benzoyl peroxide, aliphatic acids, antibiotics, salicylic acid, vitamin A derivatives, tretinoin, isotretinoin or adapalene. Treatment with such topical compositions is normally the therapy of choice for mild to moderate acne infections. Serious infections may require a longer course of treatment ranging anywhere from a week up to several months.

One ingredient used in topical preparations for acne is Clindamycin. Clindamycin is an antibiotic of the lincosamide class. Clindamycin phosphate is a water soluble ester of the semi-synthetic antibiotic produced by a 7(S)-chloro-substitution of the 7(R)-hydroxyl group of the parent antibiotic lincomycin.

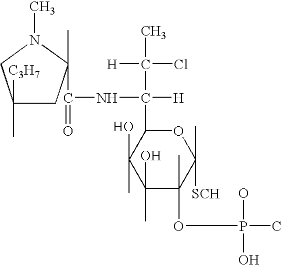

Although clindamycin phosphate is inactive in vitro, rapid in vivo hydrolysis converts this compound to the antibacterially active clindamycin. Cross resistance has been demonstrated between clindamycin and lincomycin.

Another active ingredient in treating acne is adapalene, also known as 6-[3-(1-adamantyl)-4-methoxyphenyl]-2-naphthoic acid. The molecular formula of adapalene is C₂₈H₂₈O₃ having a molecular weight of 412.52 and is represented by the following structural formula:

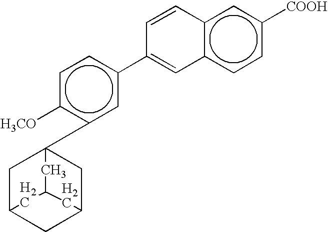

Adapalene is a chemically stable, retinoid-like and pharmacological profile studies have demonstrated that adapalene is a modulator of cellular differentiation, keratinization, and inflammatory processes all of which represent important Claims in the pathology of acne vulgaris. Mechanistically, adapalene binds to a specific retinoic acid nuclear receptor but does not bind to the cytosolic receptor protein. Although the exact mode of action of adapalene is unknown, it is suggested that topical adapalene may normalize the differentiation of follicular epithelial cells resulting in decreased microcomedone formation.

The various bacteria associated with acne have a tendency to develop resistance to solo therapy comprising either an antibiotic or a retinoid-like compound during prolonged treatment and may be rendered ineffective when the acne condition is severe. See, e.g., Weiss et al., Topical Retinoid and Antibiotic Combination Therapy for Acne Management, J. Drugs Dermatology, J. Drug Dermatology. April-May 2004: vol. 3, pp. 145-154. Weiss et al. further disclose a therapy wherein the combination of adapalene 0.1% gel plus clindamycin 0.1% gel was evaluated in a 12-week randomized trial. The subjects received either clindamycin lotion plus adapalene or clindamycin lotion plus a gel vehicle where the clindamycin was applied twice daily and the adapalene or gel vehicle once daily.

Accordingly, there remains a need for an improved pharmaceutical composition which can effectively treat all types of acne including a severe case of acne.

### SUMMARY OF THE INVENTION

One aspect of the present invention is to provide a topical dosage form of an anti-acne and antibiotic-containing pharmaceutical composition which provides release of the active pharmaceutical ingredients to the site of absorption or action to treat a skin disorder such as acne.

Another aspect of the present invention provides for processes for preparing topical drug delivery systems which release a therapeutically effective amount of one or more active pharmaceutical ingredients to the site of absorption or action.

Accordingly, in one embodiment of the present invention, a topical pharmaceutical composition is provided comprising (a) a therapeutically effective amount of at least one antiacne agent and at least one antibiotic agent as active pharmaceutical ingredients, and (b) a hydrophilic matrix capable of providing a constant and uniform release of the active pharmaceutical ingredients.

In accordance with another embodiment, a method of treating a skin disorder, e.g., a dermatological disorder, in a mammal is provided comprising topically administering to a mammal in need of such treatment a therapeutically effective amount of a topical pharmaceutical composition comprising (a) a therapeutically effective amount of at least one antiacne agent and at least one antibiotic agent as active pharmaceutical ingredients, and (b) a hydrophilic matrix capable of providing a constant and uniform release of the active pharmaceutical ingredients.

The present invention provides several advantages including at least:

1. Improved Compliance

2. Synergism

3. Enhanced Efficacy

4. Reduction of side effects

5. Economical

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a subject for treating a state, disorder, condition or causing an action is sufficient to effect such treatment or action. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include, but are not limited to, the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

The term “acne” as used herein shall be understood to mean a common inflammatory disease of the pilosebaceous glands characterized by, for example, comedones, papules, pustules, inflamed nodules, superficial pus-filled cysts, and (in extreme cases) canalizing and deep, inflamed, sometimes purulent sacs. Types of acne within the scope of the present inventive subject matter include acne vulgaris or topical acne. “Acne” is caused by an interaction among hormones, keratin, sebum, and bacteria. One common bacterial causative agent is Propionibacterium acnes.

The term “viscolising agent” as used herein is intended to mean an agent who which helps for increasing the viscosity of the final formulation and helps in building the final consistency to the final formulation. In contact with water it undergoes the polymerization and swells and forms the semisolid mass. The viscosity built up is dependent upon the degree of polymerization and the ability of the polymer to swell.

The term “alkalizing agent” as used herein is intended to mean a compound used to modify pH. In the present invention, sodium hydroxide can be used as an alkalizing agent which imparts alkalinity to, for example, the carbomer dispersion to form, e.g., a gel structure.

The term “chelating agent” as used herein is intended to mean a compound used which forms complex with metal ions and thus prevents the unwanted chemical reactions catalised by these ions. Such compounds include, by way of example and without limitation, disodium edetate and the like.

Propylene glycol is used herein in the topical formulations of the present invention as a solvent to at least disperse the active drug, adapalene. Propylene glycol is chemically stable and does not support microbial growth. Topical formulations prepared with propylene glycol do not dry on skin readily after application.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention provides for topical pharmaceutical compositions containing at least a therapeutically effective amount of at least one antiacne agent and at least one antibiotic agent as active pharmaceutical ingredients and a hydrophilic matrix having cohesion or gelling properties and capable of providing a constant and uniform release of the active pharmaceutical ingredient. The topical pharmaceutical compositions of the present invention are useful in delivering a combination of active pharmaceutical ingredients for treating a skin disorder or condition such as acne.

Suitable antiacne and antibiotic agents for use as the active pharmaceutical ingredients in the pharmaceutical compositions herein include, for example, those disclosed in Remington's Pharmaceutical Sciences, 16th Ed., 1980; Mack Publishing Co., Easton, Pa. and in Goodman and Gilman's The Pharmacological Basis of Therapeutics by Hardman and Limbird, 9th Ed., 1996, McGraw-Hill, N.Y, the contents of which are incorporated by reference herein. Examples of antiacne agents for use herein include, but are not limited to, retinoids such as adapalene, isotretinoin, tretinoin, pharmaceutically acceptable salts and esters thereof and the like and mixtures thereof. Examples of antibiotic agents for use herein include, but are not limited to, macrolids such as azithromycin, clarithromycin, lincomycin, clindamycin, erythromycin, clindamycin, pharmaceutically acceptable salts and esters thereof and the like and mixtures thereof. For example, clindamycin, an antibiotic agents also known as methyl 7-chloro-6,7,8-trideoxy-6-(1-methyl-trans-4-propyl-L-2-pyrrolidinecarboxamido)-1-thio-L-threo-α-D-galacto-octo-pyranoside or methyl 7-chloro-6,7,8-trideoxy-6-[[(1-methyl-4-propyl-2-pyrrolidinyl)carbonyl]amino]-1-thio-L-threo-α-D-galacto-octo-pyranoside, can include clindamycin as a free base or as a pharmaceutically acceptable salt and ester thereof. Examples of pharmaceutically acceptable salts and esters of clindamycin include clindamycin hydrochloride, clindamycin phosphate, clindamycin palmitate, clindamycin palmitate hydrochloride and the like. Generally, the antiacne agent will be present in the pharmaceutical compositions of the present invention in an amount ranging from about 0.01 wt. % to about 0.2 wt. % and preferably from about 0.015 wt. % to about 0.1 wt. % and the antibiotic agent will be present in the pharmaceutical compositions in an amount ranging from 0.5 wt. % to about 5 wt. % and preferably from about 0.8 wt. % to about 1.5 wt. %, based on the total weight of the composition.

In general, the hydrophilic matrix for use in the pharmaceutical compositions of the present invention advantageously provide semi solid consistency to the active pharmaceutical ingredients with enhanced cohesion or gelling properties which are pH dependant. The hydrophilic matrix generally include one or more water soluble, hydrophilic, high molecular weight, polymers having hydrogen bonding functionality and good biocompatibility. The cohesion or gelling properties of these polymers are the result of the polymer chains. The chemical nature of these polymers, including chain and side groups and crosslinking agents, generates interactions between the mucosal constituents and the polymer or polymers, such as physical entanglement, Van der Waals interactions, and hydrogen bonding. Thus, the long linear chain structure allows them to form a strong interpenetrating network with mucus and help in effective spreading on the mucosal surface uniformally.

In one embodiment, the hydrophilic matrix of the present invention includes at least a polyalkylene oxide having a weight average molecular weight of at least about 100,000, preferably a weight average molecular weight of at least about 500,000, more preferably a weight average molecular weight ranging from about 1,000,000 to about 10,000,000 and most preferably a weight average molecular weight of from about 2,000,000 to about 6,000,000. Representative examples of such polyalkylene oxides include polyethylene oxide, polypropylene oxide and the like and mixtures thereof.

In another embodiment, the hydrophilic matrix can include carbomers. Generally, carbomers are high molecular weight water-soluble polymers of acrylic acid cross-linked with allyl ethers of sucrose and/or pentaerythritol. Carbomers have different viscosities depending on their polymeric composition. Examples of carbomers for use herein include various grades of Carbopol such as, for example, Carbopol 910, 934, 940, 941, 974, 980, 981, 1342, 5984, ETD2020, ETD 2050, and Ultrez 10 (available from Noveon of Cleveland, Ohio). Generally, the hydrophilic matrix will be present in the topical compositions of the present invention in an amount ranging from about 0.1 wt. % to about 5 wt. % and preferably from about 0.2 wt. % to about 1 wt. %, based on the total weight of the composition.

The pharmaceutical composition of the present invention containing the active pharmaceutical ingredients and hydrophilic matrix can further include one or more pharmaceutically acceptable excipients. Suitable pharmaceutically acceptable excipients for use herein include, but are not limited to, fillers, viscosity builders, chelating agents, solvents (e.g., for drug dispersion), gelling agents, surfactants, buffering agents, and the like and mixtures thereof that are typically used in the art for locally applied semi-solid dosage forms. The amount of the additional pharmaceutically acceptable excipients generally varies, e.g., from about 10% to about 90% by weight, based on the total weight of the composition. The amount of the additional one or more pharmaceutically acceptable excipient generally varies, e.g., from about 10% to about 90% by weight, based on the total weight of the composition.

Fillers for use herein may be inert fillers, either water soluble or water insoluble, typically used in the pharmaceutical art for topical dosage forms. The amount of fillers varies widely, e.g., from about 1% to about 90% by weight, based on the total weight of the composition.

Viscosity builders for use herein can be any viscolising agent typically used in the pharmaceutical art for topical dosage forms. The amount of viscolising agent varies widely and will ordinarily range from about 0.1% to about 5.0% by weight, based on the total weight of the composition.

Chelating agents for use herein can be any chelating agent typically used in the pharmaceutical art for topical dosage forms. Examples for use herein includes disodium EDTA and the like and mixtures thereof. The amount of chelating agent can vary widely. For example, the amount of chelating agent can range from about 0.1% to about 5.0% by weight, based on the total weight of the composition.

Useful solvents include, but are not limited to, propylene glycol which is chemically stable and does not support the growth of micro-organism. Topical preparation prepared from this solvent does not dry up on the skin.

The pharmaceutical compositions of the present invention may also contain other ingredients that are also typically used in topical pharmaceutical compositions such as, for example, preservatives. In one embodiment, Poloxamer 407 is used as a preservative. The amount of the other ingredients varies widely and can range from about 0.1% to about 5.0% by weight, based on the total weight of the composition.

The pharmaceutical compositions of the present invention can also contain one or more neutralizers to adjust the pH of the composition. Useful neutralizers include, but are not limited to, an alkali metal hydroxide such as, for example, sodium hydroxide, potassium hydroxide, ammonium hydroxide and the like and mixtures there of. In one embodiment the neutralizer is sodium hydroxide.

The pharmaceutical compositions of the present invention are particularly useful for the treatment of dermatological ailments, conditions and afflictions having an inflammatory or proliferative component such as, for example, common acne, comedones, polymorphous acne, nodulocystic acne, acne conglobata, secondary acne such as solar, drug-related or occupational acne; widespread and/or severe forms of psoriasis, ichtyoses and ichtyosiform states; Darier's disease; actinic keratoses; palmo plantar keratoderma and keratosis pilaris; leucoplasias and leucoplasiform states, lichen planus; any benign or malignant, severe and extensive dermatological preparations.

Formulations for topical use of the pharmaceutical compositions of the present invention can be provided as a topical composition wherein the pharmacologically active ingredients are mixed with the hydrophilic matrix to form a semisolid consistency. Examples of such topical pharmaceutical compositions include, but are not limited to, a gel, cream, lotion, suspension, emulsion, ointment, foam, paste and the like. Alternatively, the topical pharmaceutical compositions of the present invention can be formulated in a semi-liquid formulation. Examples of such topical pharmaceutical compositions include, but are not limited to, a topical solution, spray, mist, drops and the like. The pharmaceutical compositions can also be administered by way of injection or a transdermal patch.

Ointments, as is well known in the art of pharmaceutical formulation, are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be readily be understood by those skilled in the field of pharmaceutical formulation, gels are semi-solid, suspension-type systems. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also can contain an alcohol and optionally an oil. In order to prepare a uniform gel, dispersing agents such as alcohol or glycerin can be added, or the gelling agent can be dispersed by tritration, mechanical mixing or stirring, or combinations thereof. The amount of gelling agents varies widely and will ordinarily range from about 0.1% to about 2.0% by weight, based on the total weight of the composition. The gel forming agent also work by the principle of copolymerization. Under alkaline pH, carbomer in presence of water undergoes cross linking and forms a gel like structure. The degree of polymerization is dependent upon the pH. At a threshold pH, the viscosities achieved by the polymer grade is the maximum.

Lotions, are preparations to be applied to the skin surface without friction, and are typically semi-liquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semi-solid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

In another embodiment of the present invention, topical dosage forms of the pharmaceutical compositions herein can be prepared by at least (a) preparation of a hydrophilic matrix phase, e.g., a carbomer phase; (b) preparation of a drug dispersion phase containing at least one or more antiacne agent(s); (c) preparation of a solution containing one or more antibiotic agents; and (d) mixing the components (a)-(c) to form a topical pharmaceutical composition. If desired, a neutralizer solution phase can also be included in the formation of the topical compositions herein.

The following example is provided to enable one skilled in the art to practice the invention and is merely illustrative of the invention. The example should not be read as limiting the scope of the claims.

### EXAMPLE 1

The ingredients for use in preparing an antiacne and antibiotic-containing topical pharmaceutical formulation in the form of a gel of this example are set forth below in Table 1. The final product was made by a cold process and incorporated the active ingredients in the product.

TABLE 1

| STEP | | Qty. | Qty. per batch (kg) |
|---|---|---|---|
| No. | Ingredients | (% w/w) | (Batch size: 600 kg) |
| I | Purified Water | 66.67 | 400.00 |
| | Disodium Edetate | 0.05 | 0.300 |
| | Carbomer 940 (Carbopol 940) | 0.55 | 3.300 |
| II | Purified Water | 0.333 | 2.00 |
| | Sodium Hydroxide | 0.078 | 0.468 |
| III | Propylene Glycol | 6.667 | 40.00 |
| | Methyl Hydroxybenzoate | 0.100 | 0.600 |
| | (Methylparaben) | | |
| | Poloxamer 407 | 0.100 | 0.600 |
| | (Lutrol F 127/Cresmer PEF 127) | | |
| | Phenoxyethanol | 0.250 | 1.500 |
| IV | Propylene Glycol | 1.333 | 8.000 |
| | Adapalene (Particle size D₉₀: | 0.105* | 0.630 |
| | 1-10 microns) | | |
| | Purified Water (rinsing) | 4.167 | 25.00 |
| V | Purified Water | 18.00 | 108.00 |
| | Clindamycin Phosphate | 1.26* | 7.56 |
| | equivalent to Clindamycin | 1.0 | — |
| | Purified Water(rinsing) | 0.833 | 5.000 |

*includes 5% overages.

Manufacturing Process:

I. Preparation of Carbomer Phase

Into a 750 liter manufacturing vessel equipped with a stirrer and filter (200# s.s. sieve) was added purified water and disodium edetate. Next, Carbomer 940 (Carbopol 940) was added slowly under stirring and allowed to soak for 1 hour.

II. Preparation of Preservatives and Drug Dispersion Phase

Into a 50 liter vessel was added 40 kg propylene glycol. The vessel was heated with the aid of steam up to 60° C. to 62° C. Under stirring, methyl hydroxybenzoate (methylparaben) and Poloxamer 407 (Lutrol F 127/Cresmer F 127), was added until dissolved. The mixture was cooled to 45° C., and 1.5 kg phenoxyethanol was added.

Into a 25 liter vessel was added 8.0 kg propylene glycol, and adapalene (micronised) was dispersed under stirring. The adapalene dispersion was passed through a 100 # ss sieve to the 50 liter vessel under continued stirring for 10 minutes to form a uniform dispersion.

III. Preparation of Sodium Hydroxide Solution Phase

Into a 5 liter ss vessel was added 2.0 kg purified water. Under stirring, sodium hydroxide was then dissolved in the water.

IV. Preparation of Gel

The sodium hydroxide solution of step III was transferred under continuous stirring for 10 minutes to 12 minutes to the vessel of step I to form an opaque semi-solid gel.

V. Preparation and Addition of Clindamycin Phosphate Solution

Into a 400 liter ss bowl was added 108.0 kg purified water. Under stirring, clindamycin phosphate was added for 10 minutes until it was dissolved completely. The mixture was transferred slowly under stirring to the components of steps II and IV (bulk) and rinsed with purified water.

VI. Mixing

The bulk was mixed for 30 minutes at slow speed. The pH was checked at 25° C.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A topical pharmaceutical composition comprising (a) a therapeutically effective amount of adapalene or a pharmaceutically acceptable salt or ester thereof; (b) clindamycin or a pharmaceutically acceptable salt or ester thereof: and (c) a hydrophilic matrix capable of providing a constant and uniform release of the active pharmaceutical ingredients.

2-11. (canceled)

12. The topical pharmaceutical composition of claim 1, wherein the hydrophilic matrix comprises a polyalkylene oxide having a weight average molecular weight of at least about 100,000.

13. The topical pharmaceutical composition of claim 1, wherein the hydrophilic matrix comprises a polyalkylene oxide having a weight average molecular weight of at least about 500,000.

14. The topical pharmaceutical composition of claim 1, wherein the hydrophilic matrix comprises a polymer of acrylic acid cross-linked with allyl ethers of sucrose and/or pentaerythritol.

15. The topical pharmaceutical composition of claim 1, wherein the hydrophilic matrix comprises a carbopol.

16. The topical pharmaceutical composition of claim 1, wherein the hydrophilic matrix is present in an amount of from about 0.1 wt. % to about 5 wt. %.

17. The topical pharmaceutical composition of claim 1, further comprising at least one pharmaceutically acceptable excipient selected from the group consisting of a filler, viscosity builder, chelating agent, solvent, gelling agent, surfactant, buffering agent and mixtures thereof.

18. (canceled)

19. The topical pharmaceutical composition of claim 1, which is a gel, cream, lotion, suspension, emulsion, ointment, foam or paste.

20. (canceled)

21. A process for preparing a topical pharmaceutical composition comprising (a) preparing a drug dispersion phase comprising a therapeutically effective amount of at least one or more antiacne agent(s); (b) preparing a solution comprising a therapeutically effective amount of one or more antibiotic agents; (c) preparing a hydrophilic matrix phase capable of providing a constant and uniform release of the at least one antiacne agent and at least one antibiotic agent; and (d) mixing components (a)-(c) to form a topical pharmaceutical composition.

22. The process of claim 21, wherein the antiacne agent is adapalene or a pharmaceutically acceptable salt or ester thereof and the antibiotic agent is clindamycin or a pharmaceutically acceptable salt or ester thereof.

23. The process of claim 22, wherein the antiacne agent is present in an amount of from about 0.01 wt. % to about 0.2 wt. % and the antibiotic agent is present in an amount of from about 0.5 wt. % to about 5 wt %.

24. (canceled)

25. The process of claim 21, wherein the hydrophilic matrix phase comprises a polyalkylene oxide having a weight average molecular weight of at least about 100,000.

26. The process of claim 21, wherein the hydrophilic matrix phase comprises a polymer of acrylic acid cross-linked with allyl ethers of sucrose and/or pentaerythritol.

27. The process of claim 21, wherein the hydrophilic matrix phase comprises a carbopol.

28. The process of claim 21, wherein the hydrophilic matrix phase is present in an amount of from about 0.1 wt. % to about 5 wt. %.

29. The process of claim 21, further comprising at least one pharmaceutically acceptable excipient selected from the group consisting of a filler, viscosity builder, chelating agent, solvent, gelling agent, surfactant, buffering agent and mixtures thereof.

30. (canceled)

31. The process of claim 21, wherein the topical pharmaceutical composition is a gel, cream, lotion, suspension, emulsion, ointment, foam or paste.

32. (canceled)

33. The process of claim 21, further comprising the step of adding a neutralizer solution phase to adjust the pH of the mixture.

34. (canceled)

35. The topical pharmaceutical composition of claim 1, wherein the adapalene or a pharmaceutically acceptable salt or ester thereof is present in an amount of from about 0.01 wt. % to about 0.2 wt. % and the clindamycin or a pharmaceutically acceptable salt or ester thereof is present in an amount of from about 0.5 wt. % to about 5 wt %.

36. (canceled)

37. A method of treating a patient having a dermatological condition which comprises administering to the patient the topical composition of claim 1.
